(19) United States
(12) Patent Application Publication
(10) Pub. No.: US 2009/0306137 A1
(43) Pub. Date: Dec. 10, 2009

(54) TREATMENT FOR DEPRESSIVE DISORDERS

(76) Inventors: Curt D. Wolfgang, Germantown, MD (US); Mihael H. Polymeropoulos, Potomac, MD (US)

Correspondence Address: HOFFMAN WARNICK LLC, 75 STATE STREET, 14TH FLOOR, ALBANY, NY, 12207, US

(21) Appl. No.: 12/301,675

(22) PCT Filed: May 21, 2007
(86) PCT No.: PCT/US07/69373

§ 371 Date: Nov. 20, 2008

Related U.S. Application Data
(60) Provisional application No. 60/747,861, filed on May 22, 2006.

(51) Int. Cl. A61K 31/454; A61P 25/24

(52) U.S. Cl. 514/321

(57) ABSTRACT

A method of treating depression comprising administering a melatonin agonist.

### CROSS-REFERENCE TO RELATED APPLICATIONS

This application claims the benefit of co-pending US Provisional Patent Application No. 60/747,861, filed 22 May 2006, which is hereby incorporated herein.

### BACKGROUND OF THE INVENTION

1. Field of the Invention

This invention is in the field of drug therapy for depressive illnesses.

2. Related Art

Depressive disorders affect nearly 20 million adults in the U.S. alone. Left untreated, depressive disorders can be debilitating, emotionally as well as physically.

Depressive disorders comprise an array of symptoms, which are listed in a booklet published by the U.S. National Institute of Mental Health (NIMH), entitled, “Depression,” as follows:

“Persistent sad, anxious, or “empty” mood

Feelings of hopelessness, pessimism

Feelings of guilt, worthlessness, helplessness

Loss of interest or pleasure in hobbies and activities that were once enjoyed, including sex

Decreased energy, fatigue, being “slowed down”

Difficulty concentrating, remembering, making decisions

Insomnia, early-morning awakening, or oversleeping

Appetite and/or weight loss or overeating and weight gain

Thoughts of death or suicide; suicide attempts

Restlessness, irritability

Persistent physical symptoms that do not respond to treatment, such as headaches, digestive disorders, and chronic pain.”

According to the NIMH booklet, three of the most common types of depressive illness are:

“Major depression is manifested by a combination of symptoms (see symptom list) that interfere with the ability to work, study, sleep, eat, and enjoy once pleasurable activities. Such a disabling episode of depression may occur only once but more commonly occurs several times in a lifetime.

A less severe type of depression, dysthymia, involves long-term, chronic symptoms that do not disable, but keep one from functioning well or from feeling good. Many people with dysthymia also experience major depressive episodes at some time in their lives.

Another type of depression is bipolar disorder, also called manic-depressive illness. Not nearly as prevalent as other forms of depressive disorders, bipolar disorder is characterized by cycling mood changes: severe highs (mania) and lows (depression). Sometimes the mood switches are dramatic and rapid, but most often they are gradual. When in the depressed cycle, an individual can have any or all of the symptoms of a depressive disorder. When in the manic cycle, the individual may be overactive, overtalkative, and have a great deal of energy. Mania often affects thinking, judgment, and social behavior in ways that cause serious problems and embarrassment. For example, the individual in a manic phase may feel elated, full of grand schemes that might range from unwise business decisions to romantic sprees. Mania, left untreated, may worsen to a psychotic state.”

### SUMMARY OF THE INVENTION

The method of the invention comprises treatment or prevention of major depression, obsessive-compulsive disorder, panic disorder, social anxiety disorder, social phobia, post-traumatic stress disorder, premenstrual dysphoric disorder, postpartum depression, major depression, dysthymia, treatment-resistant major depression, treatment-resistant bipolar disorder, and generalized anxiety disorder, or one or more symptoms thereof.

### DETAILED DESCRIPTION

Iloperidone (1-[4-[3-[4-(6-flouro-1,2-benzisoxazol-3-yl)-1-piperidinyl]propoxy]-3-methoxyphenyl]ethanone) is disclosed in U.S. Pat. No. 5,364,866, which is incorporated herein by reference. Metabolites of Iloperidone, e.g., P88 (also referred to as P-88-8891), are useful in the present invention. See, e.g., WO03020707, which is incorporated herein by reference. In some cases, it may be advantageous to use iloperidone preferentially in patients with certain genotypes as disclosed, e.g., in WO2006039663 and in WO2003054226, which are incorporated herein by reference.

Iloperidone metabolites include: 4-[3-[4-(6-Fluoro-1,2-benzisoxazol-3-yl)-1-piperidinyl]propoxy]-3-methoxy-α-methylbenzenemethanol, 1-[4-[3-[4-(6-Fluoro-1,2-benzisoxazol-3-yl)-1-piperidinyl]propoxy]-3-hydroxyphenyl]ethanone, 1-[4-[3-[4-(6-Fluoro-1,2-benzisoxazol-3-yl)-1-piperidinyl ]propoxy]-3-methoxyphenyl]-2-hydroxyethanone, 4-[3-[4-(6-Fluoro-1,2-benzisoxazol-3-yl)-1-piperidinyl]propoxy]-3-hydroxy-α-methylbenzene methanol, hydroxy-α-methylbenzene methanol, 4-[3-[4-(6-Fluoro-1,2-benzisoxazol-3-yl)-1-piperidinyl]propoxyl-2-hydroxy-5-methoxy-α-methylbenzenemethanol, 1-[4-[3-[4-(6-Fluoro-1,2-benzisoxazol-3-yl)-1-piperidinyl]propoxy]-2-hydroxy-5-methoxyphenyl]ethanone, and 1-[4-[3-[4-(6-Fluoro-1,2-benzisoxazol-3-yl)-1-piperidinyl]propoxy]-2,5-dihydroxyphenyl]ethanone.

See, U.S. Pat. No. 5,364,866, WO93/09276 and WO95/11680, which are incorporated herein by reference.

P88, a preferred metabolite, is 1-[4-[3-[4-(6-Fluoro-1,2-benzisoxazol-3-yl)-1-piperidinyl]propoxy]-3-methoxyphenyl]ethanol.

Iloperidone has moderate to high affinity to a broad spectrum of monoamine receptors and acts as an antagonist at selected dopaminergic, serotoninergic, and adrenergic receptors. It has high (Kd<10 nM) affinity for 5-HT_{2A}, Neₐ₁, Ne_{a2c}, D₂, D₃ and 5-HT_{1A} receptors and moderate (Kd 10-100 nM) affinity for other dopaminergic, adrenergic, and serotoninergic receptors including 5-HT_{1A}.

An effective amount of iloperidone or an active metabolite thereof may be administered to a subject animal (typically a human but other animals, e.g., farm animals, pets and racing animals, can also be treated) by a number of routes. An effective amount is an amount that during the course of therapy will have a preventive or ameliorative effect on a depressive disorder or a symptom thereof. For example, an effective amount is an amount that prevents the occurrence or recurrence of symptoms of a depressive disorder to the same degree as selective serotonin re-uptake inhibitors such as fluoxetine, paroxetine, sertraline, etc.

An effective amount, quantitatively, may vary, e.g., depending upon the patient, the severity of the disorder or symptom being treated, and the route of administration. Such dose can be determined by routine studies. In general, for systemic administration, e.g., oral administration, a reference point for dosing is the dose of Iloperidone or an active metabolite thereof that is used to treat psychoses or symptoms thereof in humans, i.e., about 2 mg to about 24 mg, preferably about 16 mg to about 24 mg, of iloperidone or about 0.5 mg to about 24 mg, preferably about 12 mg to about 16 mg, of P88, when administered orally.

It will be understood that the dosing protocol including the amount of iloperidone or an active metabolite thereof actually administered will be determined by a physician in the light of the relevant circumstances including, for example, the condition to be treated, the chosen route of administration, the age, weight, and response of the individual patient, and the severity of the patient's symptoms. Patients should of course be monitored for possible adverse events.

For therapeutic or prophylactic use, Iloperidone or an active metabolite thereof will normally be administered as a pharmaceutical composition comprising as the (or an) essential active ingredient at least one such compound in association with a solid or liquid pharmaceutically acceptable carrier and, optionally, with pharmaceutically acceptable adjuvants and excipients employing standard and conventional techniques.

Pharmaceutical compositions useful in the practice of this invention include suitable dosage forms for oral, parenteral (including subcutaneous, intramuscular, intradermal and intravenous), transdermal, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. The pharmaceutical compositions may be prepared by conventional techniques appropriate to the desired preparation containing appropriate amounts of Iloperidone or an active metabolite thereof. See, for example, Remington's Pharmaceutical Sciences, Mack Publishing Company, Easton, Pa., 17th edition, 1985.

In making pharmaceutical compositions for use in the invention, the active ingredient(s) will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the composition can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

The compositions are preferably formulated in a unit dosage form, each dosage preferably containing from about 1 mg to about 24 mg of the active ingredient. The term “unit dosage form” refers to physically discrete units suitable as unitary dosages for human subjects and other mammals, each unit containing a predetermined quantity of active material calculated to produce the desired prophylactic or therapeutic effect over the course of a treatment period, in association with the required pharmaceutical carrier. So, for example, an adult patient suffering a depressive disorder could be prescribed 1-4 tablets, each having 1-24 mg of Iloperidone, to be taken once, twice or three times daily and might expect improvement in his or her condition within about one to about 12 weeks.

Iloperidone and its active metabolites can also be formulated in a controlled release form, e.g., delayed, sustained, or pulsatile release. Controlled release forms of Iloperidone and its active metabolites are disclosed, e.g., in US provisional patent application 60/750,229, filed Dec. 14, 2005, which is incorporated by reference.

For example, a controlled release formulation of the invention includes one in which: (i) iloperidone or P-88 dissolves at a rate of between about 3% and about 15% per hour, more preferably between about 4% and about 13% per hour, and most preferably between about 5% and about 7% per hour in a standard dissolution assay (e.g., an aqueous solvent at (1) pH 4.5, (2) pH 6.8 or (3) 0.1 N HCl, under ambient conditions), thereby providing a slow, substantially constant dosage of iloperidone or an active metabolite thereof over a period of between about 16 and about 24 hours. In another embodiment, Iloperidone or an active metabolite thereof is released in a pulsatile profile, e.g., to release approximately 25% of drug shortly following administration and approximately 25% of drug at more or less 2 hours, 4 hours, and 6 hours post-administration, or to release approximately 50% of drug shortly following administration and approximately 25% of drug at more or less 2 hours and 4 hours post-administration or to release approximately 50% of drug shortly following administration and approximately 25% of drug at more or less 4 hours and 6 hours post-administration.

Various formulations and methods of administering iloperidone and/or its derivatives have been described. For example, PCT Publication No. WO 2004/006886 A2 describes an injectable depot formulation comprising iloperidone crystals; microencapsulated depot formulations of iloperidone and a polyglycolide polylactide glucose star polymer are described in U.S. 20030091645; and methods for the administration of iloperidone directed toward, inter alia, eliminating or minimizing the prolongation of a corrected electrocardiographic QT (QTc) interval associated with increased concentrations of iloperidone or iloperidone derivatives are described in U.S. Provisional Application No. 60/614,798, filed Sep. 30, 2004, all of which are incorporated herein by reference.

The invention encompasses administration of iloperidone or an active metabolite thereof in combination with other agents, e.g., other CNS agents such as, but not limited to, agents in the following drug categories:

melatonin agonists

selective serotonin reuptake inhibitors (SSRIs)
5-HT_{1A} antagonists
5-HT_{1A}/β-adrenoceptor antagonist
5-HT_{1B} antagonists
5-HT_{2C} antagonists
   Selective and nonselective
5-HT_{2C} agonists
5-HT₆ agonists
α-2 adrenergic antagonists

serotonin and norepinephrine reuptake inhibitors (SNRIs)

monoamine oxidase inhibitors (MAOs)

tricyclic antidepressants (TCAs)

triple monoamine update blockers

benzodiazepines

NMDA receptor antagonists

Pyrrolinones

Benzothiadiazides

Benzoylpiperidnes

Biarylopropylsulfonamides

Metabotropic glutamate receptors (mGluRs)

GABA antagonists

NK1 antagonists

NK2 antagonists

CRF1 antagonists

Arginine vasopressin V1b antagonists

MCH receptor antagonists

NGF antagonists

BDNF antagonists

NT-3 antagonists

NT-4 antagonists

CREB antagonists

Illustrative, and not limiting, of such agents are:

melatonergic agonists: melatonin, agomelatine, (1R-Trans)-N-[[2-(2,3-dihydro-4-benzofuranyl) cyclopropyl]methyl]propan-amide, and N-[1-(2,3-dihydrobenzofuran-4-yl) pyrrolidin-3-yl]-N-ethylurea], ramelteon, 2-Phenylmelatonin, 8-M-PDOT, 2-Iodomelatonin, 6-Chloromelatonin serotonin reuptake inhibitors: paroxetine, fluoxetine, sertraline, venlaxafine, citalopram, escitalopram, fluvoxamine, trazadone, nefazodone, milnacipran, desipramine, duloxetine, YM992

SSRI/5-HT1A antagonists: WAY-100635, Pindolol

SSRI/5-HT1B antagonists: SB-224289

SSRI/5-HT2C antagonists

Selective: SB242084, RS102221

Nonselective: Ketanserin, Irindalone

SSRI/5-HT2C agonists: Org 37684, Ro 60-0175, WAY-161503, YM348, WAY-629, WAY-163909

SSRI/5-HT6 agonists: LY586713, WAY-466, WAY-1811187

α-2 adrenergic antagonists: Mirtazapine (Remeron)

triple monoamine update blockers: DOV 21,947

NMDA receptor antagonists: MK-801, Memantine, Ketamine, Felbamate, Glycine, D-serine, D-cycloserine, L-glutamatelfenprodil

Pyrrolidiones: Piracetam, Aniracetam

tricyclics: Amitriptyline Clomipramine Desipramine Dothiepin Doxepin Imipramine Lofepramine Nortriptyline Protriptyline Trimipramine Iprindole Opipramol

tetracyclics: Maprotiline Mianserin Mirtazapine Amoxapine Trazodone Nefazodone

serotonin reuptake enhancers: tianeptine,

monoamine oxidase inhibitors: Harmaline Nialamide Selegiline Isocarboxazid Iproniazid Iproclozide Moclobemide Phenelzine Toloxatone Tranylcypromine

dopamine reuptake inhibitors: Bupropion Amineptine Methylphenidate Phenmetrazine Vanoxerine

norepinephrine reuptake inhibitors: Atomoxetine Reboxetine Viloxazine Maprotiline Bupropion, Reboxetine

serotonin-norepinephrine reuptake inhibitors: Desipramine Duloxetine Milnacipran Nefazodone Venlafaxine

Benzothiadiazides: Cyclothiazide

Benzoylpiperidines: CX516, CX546

Biarylopropylsulfonamides: LY392098, LY404187, LY451646

Metabotropic glutamate receptors (mGluRs): 2-methyl-6-(phenylethynyl)-pyridine (MPEP), 3-[(2-methyl-1,3-thiazol-4-yl)ethynyl]-pyridine (MTEP), JNJ16259685, CPCOOEt, MGS0039, LY341495, LY354740, ACPT-1/L-SOP (L-serine-O-phosphate), HomoAMPA, N-pheynl-7-(hydroxyimino) cyclopropa[b]chromen-1a-carboxamide

GABA antagonists: CGP36742, CGP56433, CGP56999

NK1 antagonists: GW823296, GW679769, GW597599 (Vestipitant), R673, CP-122,721, L-759274, GR205171, L733060

NK2 antagonists: SR48968

CRF1 antagonists: DMP696, DMP904, GW876008, AAG561, TS-041, CP-154,526 (antalarmin), SSR125543, R278995/CRA0450, R121919

Arginine vasopressin V1b antagonists: SSR149415

MCH receptor antagonists: T-226296.

In some patients, it reportedly is useful to augment antidepressant treatment with lithium or triiodothyronine.

Thus, in another illustrative embodiment, the invention comprises a kit comprising one or more pharmaceutical dosage units of an anti-psychotic and one or more pharmaceutical dosage units of a antidepressant, wherein either or both of the anti-psychotic dosage unit and the antidepressant unit can also comprise, respectively, an antidepressant or an anti-psychotic, and optionally, one or more additional pharmaceutically active ingredients. In another embodiment, the invention comprises administering the anti-psychotic and the other agent or agents at different time intervals, such that an effective amount of each is maintained in the patient's bloodstream in the appropriate amounts at the appropriate times. Such kit could facilitate, e.g., administration of the anti-psychotic to be taken at different time intervals than the other agent or agents. In a related embodiment, the kit comprises pharmaceutical dosage units of one agent alone and other pharmaceutical dosage units comprising both agents. In this way, for example, the anti-psychotic could be taken alone during the day and with the other agent or agents in the evening.

When used in such combinations, the dose of each agent is expected to be approximately the same as, or less than, an effective amount of either alone. For example, each pharmaceutically active ingredient can be administered in doses that are about 20% to about 80% of the dose in which each ingredient would be administered alone.

The two (or more) agents can be administered more or less simultaneously, i.e., concomitantly (e.g., within about 0 to about 5 minutes of each other, preferably within about a minute apart, or they can be administered at different times. For example, in one aspect, the invention is a pharmaceutical composition comprising both the anti-psychotic agent and the other agent or agents. This embodiment, for example, comprises a pill or capsule having both active pharmaceutical ingredients either admixed together or having each active pharmaceutical ingredient in a discrete portion of the pill or capsule.

For example, the compositions can be formulated in a unit dosage form, each dosage containing both active ingredients. The term “unit dosage form” refers to physically discrete units suitable as unitary dosages for human subjects and other mammals, each unit containing a predetermined quantity of active material calculated to produce the desired prophylactic or therapeutic effect over the course of a treatment period, in association with the required pharmaceutical carrier. So, for example, an adult patient suffering a depressive disorder could be prescribed 1-4 tablets, to be taken once, twice or three times daily and might expect improvement in his or her condition within about one to about 12 weeks.

Unit dose forms of the invention, whether they comprise Iloperidone or an active metabolite thereof as the sole active pharmaceutical ingredient or in combination with another agent, e.g., another antipsychotic or antidepressant, can also be formulated in a controlled release form, e.g., delayed, sustained, or pulsatile release. With such form, in the case of combinations, the Iloperidone or active metabolite thereof can be released at the same or different rates and times as the other agent or agents.

While this invention has been described in conjunction with the specific embodiments outlined above, it is evident that many alternatives, modifications and variations will be apparent to those skilled in the art or are otherwise intended to be embraced. Accordingly, the embodiments of the invention as set forth above are intended to be illustrative, not limiting. Various changes may be made without departing from the spirit and scope of the invention as defined in the following claims. All patents, patent application, scientific articles and other published documents cited herein are hereby incorporated in their entirety for the substance of their disclosures.

1. A method for treating one or more of major depression, obsessive-compulsive disorder, panic disorder, social anxiety disorder, social phobia, post-traumatic stress disorder, premenstrual dysphoric disorder, postpartum depression, major depression, dysthymia, treatment-resistant major depression, treatment-resistant bipolar disorder, and generalized anxiety disorder, or a symptom thereof, in an animal comprising internally administering to the animal an effective amount of Iloperidone or an active metabolite thereof.

2. The method of claim 1, wherein the disorder is selected from the group consisting of: obsessive-compulsive disorder, panic disorder, social anxiety disorder, social phobia, post-traumatic stress disorder, premenstrual dysphoric disorder, dysthmia, and generalizeed anxiety disorder, or any combination thereof.

3. The method of claim 1, wherein the symptoms include at least one of the following: persistent sad, anxious, or empty mood; feelings of hopelessness; pessimism; feelings of guilt, worthlessness, or helplessness; loss of interest or pleasure in hobbies and activities that were once enjoyed, including sex; decreased energy, fatigue, or being slowed down; difficulty concentrating, remembering, or making decisions; insomnia, early-morning awakening, or oversleeping; appetite and/or weight loss or overeating and weight gain; thoughts of death or suicide; suicide attempts; restlessness; irritability; persistent physical symptoms that do not respond to treatment, such as headaches, digestive disorders, and chronic pain; or any combination of the preceding.

4. The method of claim 1, further comprising the administration of a second antipsychotic medication.

5. The method of claim 1, further comprising the administration of iloperidone or an active metabolite thereof in combination with an antidepressant.

6. The method of claim 5, wherein the antidepressant is selected from the group consisting of: melatonin agonists, selective serotonin reuptake inhibitors (SSRIs), 5-HT_{1A} antagonists, 5-HT_{1A}/β-adrenoceptor antagonist, 5-HT_{1B} antagonists, selective and nonselective5-HT_{2C} antagonists, 5-HT_{2C} agonists, 5-HT₆ agonists, α-2 adrenergic antagonists, serotonin and norepinephrine reuptake inhibitors (SNRIs), monoamine oxidase inhibitors (MAOs), tricyclic antidepressants (TCAs), triple monoamine update blockers, benzodiazepines, NMDA receptor antagonists, Pyrrolinones, Benzothiadiazides, Benzoylpiperidnes, Biarylopropylsulfonamides, Metabotropic glutamate receptors (mGluRs), GABA antagonists, NK1 antagonists, NK2 antagonists, CRF1 antagonists, Arginine vasopressin V1b antagonists, MCH receptor antagonists, NGF antagonists, BDNF antagonists, NT-3 antagonists, NT-4 antagonists, CREB antagonists, and combinations of any one or more of the recited classes of antidepressants.

7. The method of claim 6, wherein the antidepressant is selected from the group consisting of: melatonin, (1R-Trans)-N-[[2-(2,3-dihydro-4-benzofuranyl) cyclopropyl]methyl]propanamide, N-[1-(2,3-dihydrobenzofuran-4-yl) pyrrolidin-3-yl]-N-ethylurea], Agomelatine (Valdoxan), Ramelteon, 2-Phenylmelatonin, 8-M-PDOT, 2-lodomelatonin, 6-Chloromelatonin, fluoxetine (Prozac), paroxetine (Paxil), YM992, sertraline (Zoloft), venlafaxine (Effexor), bupropion (Wellbutrin), reboxetine (Edronax), WAY-100635, Pindolol, SB-224289, SB242084, RS102221, Ketanserin, Irindalone, Org 37684, Ro 60-0175, WAY-161503, YM348, WAY-629, WAY-163909, LY586713, WAY-466, WAY-1811187, Mirtazapine (Remeron), DOV 21,947, MK-801, Memantine, Ketamine, Felbamate, Glycine, D-serine, D-cycloserine, L-glutamate, Ifenprodil, Piracetam, Aniracetam, Cyclothiazide, CX516, CX546, LY392098, LY404187, LY451646, 2-methyl-6-(phenylethynyl)-pyridine (MPEP), 3-[(2-methyl-1,3-thiazol-4-yl) ethynyl]-pyridine (MTEP), JNJ16259685, CPCOOEt, MGS0039, LY341495, LY354740, ACPT-1/L-SOP (L-serine-O-phosphate), HomoAMPA, N-pheynl-7-(hydroxyimino) cyclopropa[b]chromen-1a-carboxamide, CGP36742, CGP56433, CGP56999, GW823296, GW679769, GW597599 (Vestipitant), R673, CP-122,721, L-759274, GR205171, L733060, SR48968, DMP696, DMP904, GW876008, AAG561, TS-041, CP-154,526 (antalarmin), SSR125543, R278995/CRA0450, R121919, SSR149415, T-226296, and combinations of any one or more of the recited antidepressants, including any active metabolites thereof.

8. The method of claim 1, wherein the antipsychotic and the antidepressant are administered by the same or a different route selected from parenteral, intravenous, intramuscular, buccal, lozenge, transdermal, and transmucosal.

9. A pharmaceutical composition for the treatment of a depressive disorder comprising an iloperidone or an active metabolite thereof in combination with another anti-psychotic or another antidepressant or with both.

10. The pharmaceutical composition of claim 9, which is in a unit dose form that comprises the iloperidone or active metabolite thereof and the other agent or agents, in amounts that are effective when administered as a single dose or when administered in multiple doses.

11. A kit comprising one or more pharmaceutical dosage units of an anti-psychotic and one or more pharmaceutical dosage units of a antidepressant, wherein either or both of the anti-psychotic dosage unit and the antidepressant unit can also comprise, respectively, an antidepressant or an anti-psychotic, and optionally, one or more additional pharmaceutically active ingredients.

12-17. (canceled)
